(19) United States
(12) Patent Application Publication
(10) Pub. No.: US 2006/0099272 A1
(43) Pub. Date: May 11, 2006

(54) Compositions and kits including dehydrated stinging capsules and methods of producing and using same

(75) Inventor: Tamar Lotan, Jordan Valley (IL)

Correspondence Address: Martin Moynihan;c/o ANTHONY CASTORINA, SUITE 207, 2001 JEFFERSON DAVIS HIGHWAY, ARLINGTON, VA, 22202, US

(73) Assignee: NanoCyte Inc.

(21) Appl. No.: 10/981,498

(22) Filed: Nov. 5, 2004

(51) Int. Cl. A61K 35/12

(52) U.S. Cl. 424/529

(57) ABSTRACT

A dry composition-of-matter and a kit including dehydrated stinging capsules and methods of producing and using same are provided.

### FIELD AND BACKGROUND OF THE INVENTION

The present invention relates to methods of producing dehydrated stinging capsules, compositions and kits including same and their use in delivering a therapeutic, diagnostic or cosmetic agent into a tissue.

Therapeutic agents such as drugs are a mainstay of modem medicine and are used for the prevention, diagnosis, alleviation, treatment, or cure of diseases.

Biological, biochemical and/or physical barriers often limit delivery of therapeutic agents to target tissue. For example, skin and/or various organ membranes are physical barriers, which must be traversed by a topically administered drug targeted at internal tissues. Orally administered drugs must be resistant to the low pH conditions and digestive enzymes present in the gastrointestinal (GI) tract.

To traverse such barriers, drugs targeted at internal tissues are often administered via a transdermal injection, using a syringe and a needle or other mechanical devices. A transdermal injection delivers drugs into the subcutaneous space thus traversing the epidermis-dermis layers.

Anatomically, the skin of a human body is subdivided into three compartments: an epidermis, a dermis and a subcutaneous layer, of which the epidermis plays a key role in blocking drug delivery via the skin (the dourest layer of the epidermis is the stratum corneum which is called also the horny layer). The epidermis is 0.1 mm or more in thickness and consists mainly of protein surrounded by lipid, thus rendering the epidermis hydrophobic.

Although the syringe and needle is an effective delivery device, it is sensitive to contamination, while use thereof is often accompanied by pain and/or bruising. In addition, the use of such a device is accompanied by risk of accidental needle injury to a health care provider.

Mechanical injection devices based on compressed gasses have been developed to overcome the above-mentioned limitations of syringe and needle devices. Such devices typically utilize compressed gas (such as, helium or carbon dioxide) to deliver medications at high velocity through a narrow aperture.

Although such devices traverses some of the limitations mentioned above, their efficiency is medication dependent, and their use can lead to pain, bruising and lacerations.

Other less common delivery methods utilize a pulsed Yag laser to punctuate the stratum corneum in order to deliver medication via diffusion and enhancement of ionic compound flux across the skin by the application of an electric current. Although such methods are effective in delivering small charged molecules, a danger of skin burns accompanies their use.

Non-invasive methods, which overcome some of the limitations inherent to the invasive delivery methods described above, have also been described. Such methods utilize preparations, which include an active ingredient disposed within lipid vehicles (e.g., liposomes) or micelles or accompanied with skin permeation agent such that absorption of the active ingredient through the skin is enhanced. Such preparations can be directly applied to a skin region or delivered via transdermal devices such as membranes, pressure-sensitive adhesive matrices and skin patches.

In transdermal delivery, the active ingredient penetrates the skin and enters the capillary blood or the lymph circulation system, which carries the drug to the target organ or to the tissue or has a local effect.

For several years, transdermal drug delivery systems have been employed to effectively introduce a limited number of drugs through unbroken skin. Aside from comfort and convenience, transdermal systems avoid the barriers, delivery rate control problems and potential toxicity concerns associated with traditional administration techniques, such as oral, intramuscular or intravenous delivery.

Although transdermal delivery offers an alternative to some invasive delivery methods, the efficiency thereof is affected by the physical and chemical properties of a drug and physiological or pathological parameters such as the skin hydration, temperature, location, injury, and the body metabolism.

Many limitations of invasive and non-invasive delivery devices may be circumvented by the use of “stinging capsules” (e.g., cnidocysts, nematocysts and polar capsules) isolated therefrom for tissue delivery of a therapeutic, diagnostic or cosmetic agents.

Cnidaria (hydras, sea anemones, jellyfish and corals) are aquatic animals, which possess a variety of compounds which are stored and delivered via specialized capsules (cnidocysts), which form a part of specialized cells termed stinging cells (cnidocytes, nematocytes, ptychocytes and the like). The stinging capsules act as microscopic syringes and serve as a prey or defense mechanism. The Cnidaria family which encompasses 10,000 known species, includes sedentary single or colonial polyps and pelagic jellyfish. In some of these species, cnidocytes account for more than 45% of the cells present (Tardent 1995).

Discharge is initiated by a rapid osmotic influx of water which generates an internal hydrostatic (liquid) pressure of 150 atmospheres forcing capsule rupture and ejection of the tubule (Holstein and Tardent 1984). During ejection, the long coiled and twisted tubule is averted and its length increases by 95 percent. Accelerating at 40,000 g, the tubule untwists to generate a torque force, which rotates the tubule several times around its axis. These mechanical processes generate a powerful driving force, which enables efficient delivery of the compounds, the toxins and enzymes stored within the capsule (Lotan et al. 1995, 1996; Tardent 1995). This process, which occurs within microseconds, is among the most rapid exocytosis events in biology (Holstein and Tardent 1984).

There are at least three dozen known types of cnidocysts (also termed cnidae) including more than 30 varieties of nematocysts found in most Cnidaria and spirocysts, and ptychocysts found mainly in the Cnidaria class Anthozoa (Mariscal 1974).

Accordingly, U.S. Pat. No. 6,163,344 and U.S. patent application Ser. Nos. 10/406,202 and 09/963,672 to the present inventors teach the use of stinging capsules or cells for the purpose of rapidly and efficiently delivering therapeutic, cosmetic or diagnostic agents into a target tissue. However, isolated stinging capsules hold moisture which hinders their stability in storage. Furthermore, the activation of moist stinging capsules is often untimely and inconsistent.

The present invention provides novel dry compositions and kits including dehydrated stinging capsules which can be stored for extended periods of time while retaining their ability to discharge upon activation. The dry compositions and kits of the present invention can be utilized for delivering therapeutic, diagnostic or cosmetic agents into a tissue effectively, rapidly and consistently.

### SUMMARY OF THE INVENTION

According to one aspect of the present invention there is provided a dry composition-of-matter which includes at least one dehydrated stinging capsule.

According to another aspect of the present invention there is provided a kit which includes a first component including at least one dehydrated stinging capsule, and a second component including a rehydration substance capable of activating the at least one dehydrated stinging capsule.

According to yet another aspect of the present invention there is provided a method of producing dehydrated stinging capsules. The method includes subjecting a stinging capsule preparation to desiccating conditions, to thereby producing the dehydrated stinging capsules.

According to still another aspect of the present invention there is provided a method of delivering a therapeutic, diagnostic or cosmetic agent into a tissue. The method includes the steps of (a) applying to the tissue at least one dehydrated stinging capsule and the therapeutic, diagnostic or cosmetic agent, and (b) activating a discharge of the at least one dehydrated stinging capsule to thereby deliver the therapeutic, diagnostic or cosmetic agent into the tissue.

According to further features in preferred embodiments of the invention described below, the moisture content of the dry composition-of-matter is less than 10% by weight.

According to still further features in the described preferred embodiments the dry composition-of-matter further includes a therapeutic, diagnostic or cosmetic agent.

According to still further features in the described preferred embodiments the therapeutic, diagnostic or cosmetic agent is disposed within the dehydrated stinging capsule(s).

According to still further features in the described preferred embodiments the cosmetic agent is selected from the group consisting of a cosmetic dye, an anti wrinkling agent, an anti-acne agent, a vitamin, a skin peel agent, a hair follicle stimulating agent and a hair follicle suppressing agent.

According to still further features in the described preferred embodiments the therapeutic agent is selected from the group consisting of a drug, a nucleic acid construct, a vaccine, a hormone, an enzyme and an antibody.

According to still further features in the described preferred embodiments the therapeutic agent is a prodrug activatable prior to, during or following discharge of the at least one dehydrated stinging capsule.

According to still further features in the described preferred embodiments the at least one dehydrated stinging capsule is capable of delivering the therapeutic, diagnostic or cosmetic agent into a tissue.

According to still further features in the described preferred embodiments an endogenous toxin naturally stored within the dehydrated stinging capsule(s) is substantially non-toxic to mammals.

According to still further features in the described preferred embodiments the endogenous toxin is non-functional.

According to still further features in the described preferred embodiments the dehydrated stinging capsule is derived from an organism of a class selected from the group consisting of Anthozoa, Hydrozoa and Scyphozoa.

According to still further features in the described preferred embodiments the dehydrated stinging capsule is derived from an organism of a phylum selected from the group consisting of Cnidaria, Dinoflagellata and Myxozoa.

According to still further features in the described preferred embodiments the rehydration substance is a buffer solution.

According to still further features in the described preferred embodiments the rehydration substance includes at least one triggering agent.

According to still further features in the described preferred embodiments the kit includes a third component including a therapeutic, diagnostic or cosmetic agent.

According to still further features in the described preferred embodiments, the desiccation conditions include exposing stinging capsules to a substance capable of absorbing moisture from the stinging capsules.

According to still further features in the described preferred embodiments the s kit includes an instructions-for-use leaflet and a packaging material which identifies the kit for use in delivering a therapeutic, diagnostic or cosmetic agent to a tissue.

According to still further features in the described preferred embodiments the desiccating conditions include freezing the stinging capsule preparation.

According to still further features in the described preferred embodiments the freezing is effected under vacuum conditions.

According to still further features in the described preferred embodiments, prior to subjecting the stinging capsule preparation to the desiccating conditions, the stinging capsules are exposed to a substance capable of absorbing moisture from the stinging capsules.

According to still further features in the described preferred embodiments the substance capable of absorbing moisture is selected from the group consisting of ethanol, propylene glycol, acetyl palmitate and a salt.

According to still further features in the described preferred embodiments the step of activating a discharge of the dehydrated stinging capsule(s) is effected by exposing the dehydrated stinging capsule(s) to a rehydration substance.

The present invention successfully addresses the shortcomings of the presently known configurations by providing compositions and kits including dehydrated stinging capsules which are highly stable in storage which can be utilized for safe, convenient and rapid delivery of therapeutic, diagnostic or cosmetic agents into a tissue.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates freeze-dried stinging capsules of sea anemone which have been suspended in propylene glycol. The FIGURE shows that the capsules are intact and readily dispersed without forming aggregates.

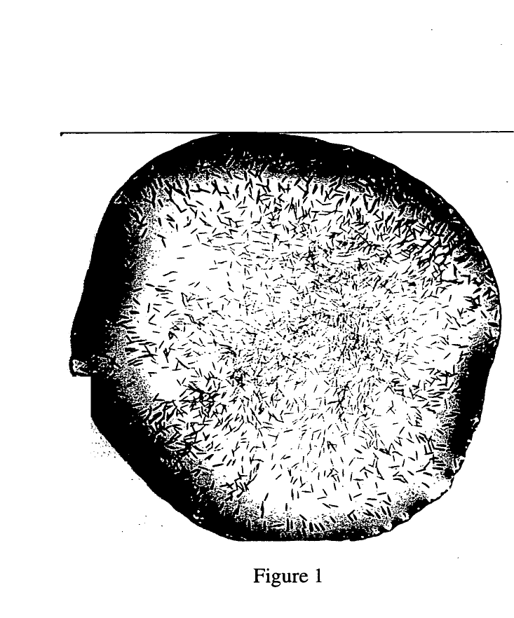

### DESCRIPTION OF THE PREFERRED EMBODIMENTS

The present invention is of compositions and kits including dehydrated stinging capsules (cnidocysts) and methods of producing and utilizing same for delivery of an agent to a tissue.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As described in the background section above, U.S. Pat. No. 6,163,344 and U.S. patent application Ser. Nos. 10/406,202 and 09/963,672 teach use of stinging capsules (cnidocysts) or cells for delivering therapeutic, diagnostic or cosmetic agents into a tissue.

The cnidocyst is filled with liquid containing a highly folded inverted tubule. In nature, the cnidocyst discharges and releases its tubule into tissue following physical or chemical triggering. Discharge is initiated by a rapid osmotic influx of water which generates an internal hydrostatic (liquid) pressure of 150 atmospheres forcing capsule rupture and ejection of the tubule (Holstein and Tardent 1984). During ejection, the long coiled and twisted tubule is averted and its length increases by 95 percent. Accelerating at 40,000 g, the tubule untwists to generate a torque force, which rotates the tubule several times around its axis. These mechanical processes generate a powerful driving force, which enables efficient delivery of the natural compounds, (toxins and enzymes) stored within the capsule (Lotan et al. 1995, 1996; Tardent 1995). This process, which occurs within microseconds, is among the most rapid exocytosis events in biology (Holstein and Tardent 1984).

U.S. Pat. No. 6,163,344 and U.S. patent application Ser. Nos. 10/406,202 and 09/963,672 describe compositions which utilize stinging capsules or cells for delivering pharmaceutical or cosmetic agents into a tissue. While stinging capsules hold considerably less moisture than stinging cells, the moisture content thereof is still high enough to negatively affect their stability in long-term storage. It would therefore be highly desired to produce dehydrated stinging capsule compositions which could be stored for extended periods without lose of activity. However, since stinging capsules have a highly complex, delicate and trigger-sensitive system, as described hereinabove, it was not expected that stinging capsules would remain viable following desiccation.

While reducing the present invention to practice, the present inventors surprisingly and unexpectedly uncovered that stinging capsules can be effectively dehydrated and that the dehydrated capsules remain highly stable in storage while retaining their full discharging capacity following rehydration.

Thus, according to one aspect of the present invention there is provided a dry composition-of-matter which includes at least one dehydrated stinging capsule.

The stinging capsule of the present invention can be derived from an organism of the phylum Cnidaria, Myxozoa, or Dinoflagellata, preferably it is derived from an organism of the class Anthozoa, Hydrozoa or Scyphozoa. More specifically, the stinging capsule utilized by the present invention can be derived from, for example, subclasses Hexacorallia or Octocorallia of the class Anthozoa, (mostly sea anemone and corals), subclasses Siponophora or Hydroida of the class Hydrozoa, or from subclasses Rhisostomeae or Semastomeae of the class Scyphozoa.

Stinging capsules from such organisms include toxins, which are non-toxic to humans, and other mammals. As such, these stinging capsules isolated therefrom are ideally suited for safe and efficient delivery of a therapeutic, diagnostic or cosmetic agent into mammalian tissue.

It will be appreciated that the use of stinging capsules from organisms which sequester toxins that are not fatal but cause only minor irritations to, for example, mammals, is also envisioned by the present invention.

The stinging capsule of the present invention can be isolated from a cell extract prepared from organs or parts of an organism, which contain the stinging cells (for example a whole hydra or tentacles). Alternatively, stem cells, which give rise to cnidocytes or cnidocysts, can be isolated and cultured or utilized directly.

In addition, stinging capsules from other sources can also be utilized by the present invention provided inactivation of the endogenous toxin is effected prior to use. Such inactivation can be effected via one of several methods such as described in U.S. Pat. No. 6,613,344.

As used herein the phrase “dehydrated stinging capsule” refers to a stinging capsule which is substantially devoid of any water based substance liquid (e.g., water). Thus, preferably, the dry composition-of-matter of the present invention has a moisture content of less than 10% w/w.

Several approaches can be used to produce dehydrated capsule compositions. Preferably, the dehydrated stinging capsule of the present invention can be produced via a lyophilization (freeze-drying) procedure. The procedure involves suspending the capsules in a solution, freezing the capsules suspension then subjecting the frozen suspension to sublimation under vacuum.

Freeze-drying is a routine technique used in the art (see, for example, in “A Guide to Freeze-Drying for the Laboratory”, An Industry Service Publication, Labcono, Kansas City, Mo. USA, 1998; Frank, F. (Ed.), Effective freeze-drying: a combination of physics, chemistry, engineering and economics. Proc. Inst. Refrigeration 91: 32-39; Rey, J. and May, C. (Eds), Freeze-drying/lyophilization of pharmaceutical and biological products. C.H.I.P.S. Press, 1999; Costantino, H. R. and J. Pikal. (Eds.), Lyophilization of Biopharmaceuticals. AAPS Press, 2004).

Initially the isolated stinging capsules are suspended in a water solution or, preferably, a freezing buffer. The freezing buffer preferably including a cryoprotective agent such as, but not limited to, glucose, sucrose, lactose, manitol, glycerol, dextran, fructose, monosodium glutamate, polyvinylpyrrolidone (PVP), sweet whey solids, dried skim milk and bovine serum albumin. The stinging capsule suspension is then dispensed in suitable containers (e.g., Eppendorf tubes or standard glass vials covered with rubber stoppers) and pre-frozen below the glass transition point (Tg), preferably in liquid nitrogen or in a −80° C. freezer. The containers are then placed in a vacuum chamber and subjected to a reduced pressure, preferably under 1 mbar, while the temperature in the chamber is raised to just below the Tg point to effect the freeze-drying process. Following sublimation of the frozen water, drying continues at a higher temperature in order to evaporate any residual moisture.

As is illustrated in Example 1 of the Examples section which follows, freeze dried stinging capsule preparations which were stored for one year at 4° C., or for six month at room temperature, or for one month at 40° C., remained capable of discharging tubules immediately upon rehydration.

Alternatively, dehydration of the stinging capsules of the present invention can be effected by desiccation of non-frozen capsules.

Prior to desiccation, the capsules are preferably exposed to a substance capable of absorbing water from capsules such as an organic solvent (e.g., ethanol (70% or 100%), propylene glycol or octyl palmitate). More specifically, the capsules are preferably suspended in the organic solvent, incubated for a period of time (ranging from several minutes to several days) followed by centrifugation and discarding the supernatant. The process may be repeated for several times and the resulting pellet is then subjected to desiccation which can be effected under vacuum condition (with or without silica gel) or under a fume or laminar flow hood and the like.

Alternatively or additionally, prior to desiccation, the capsules may be exposed to a hygroscopic salt such as, but not limited to, CaCl₂, MgSO₄ or NaSO₄.

The dehydrated stinging capsules are preferably formulated in a dry formulation such as, for example, a powder, granular or tablet formulation or in a semi-solid, a liquid or a spray formulation.

As is illustrated in Example 2 of the Examples section which follows, desiccated non-frozen stinging capsules retained their discharging capacity following storage at 4° C. for a period of at least 1 month.

The stinging capsules can be modified to include an agent prior to desiccation, via any one of several methods generally known in the art such as, but not limited to, diffusion, electroporation, liposome fusion, microinjection and the like.

Prior art studies which concentrated on deciphering the permeability and functionality of stinging capsules have shown that alkali ions, monovalent ions, divalent ions, or small organic cations such as Tris+ or choline+, penetrate cnidocysts and accumulate inside without affecting the properties of the stinging capsule. Studies performed by Lubbock & Amos in order to understand the effect of calcium on capsule discharge (1981) have shown that in the pre-discharged state the cnida wall is permeable to water and to charged molecules of relatively low molecular weight like bromophenol blue (MW 670) and fluoresceinate (MW 376). Hidaka, who investigated of the mechanism of capsule discharge (1992, 1993) demonstrated that cnidocysts stained with toluidine blue (MW 306) released the blue stain through the tubule when discharged leaving the capsule completely clear. Heeger et al., (1992) investigated the ability of different commercially available lotions to protect human skin against stinging capsules.

Thus, short polypeptides, hormones, or any low molecule weight agents can be loaded into stinging capsules through simple diffusion. These active compounds can be stored in the capsule and injected into the target tissue upon discharge.

Thus, the dehydrated stinging capsules of the present invention are highly stable in storage and remain suitable for delivery of therapeutic, diagnostic or cosmetic agents into a host tissue.

Delivery of a therapeutic, diagnostic or cosmetic agent into a tissue can be effected by applying the agent to an outer surface of the tissue (e.g., skin) which is preferably surface sterilized with a suitable solution (e.g., alcohol). The stinging capsules are then exposed to a rehydration substance which activates the stinging capsules. Following activation, the stinging capsules deliver the therapeutic, diagnostic or cosmetic agent via the tubules into the tissue.

The dehydrated stinging capsule composition and the therapeutic, diagnostic or cosmetic agent can be applied to the outer surface of the tissue either sequentially (as separate compositions) or concomitantly by having the agent pre-disposed within the dehydrated capsules, as described hereinabove.

As used herein, the phrase “rehydration substance” refers to any substance which is capable of activating dehydrated stinging capsules such as, for example, an aqueous solution, a spray, a foam, a gel, a paste, a cream or a semi-solid. Preferably, the rehydration substance is a buffer solution, more preferably a saline solution. As further described hereinbelow, the rehydration substance of the present invention may further include one or more triggering agents. Alternatively, or additionally, the rehydration substance can also include the therapeutic, cosmetic or diagnostic agent being dissolved, or suspended, within the formulation.

According to preferred embodiments of the present invention, the therapeutic agent can be any biological active factor such as, for example, a drug, a nucleic acid construct, a vaccine, a hormone, an enzyme, small molecules such as for example iodine or an antibody. Examples include, but are not limited to, antibiotic agents, free radical generating agents, anti fungal agents, anti-viral agents, non-nucleoside reverse transcriptase inhibitors, protease inhibitors, non-steroidal anti inflammatory drugs, immunosuppressants, anti-histamine agents, retinoid agents, tar agents, anti-puritic agents, hormones, psoralen, and scabicide agents. Nucleic acid constructs deliverable by the present invention can encode polypeptides (such as enzymes ligands or peptide drugs), antisense RNA, or ribozymes.

The therapeutic agent can also be a prodrug, which is activatable prior to, during, or following discharge of the stinging capsule. As used herein in the specification and in the claims section which follows, the term “prodrug” refers to an agent which is inactive but which is convertible into an active form via enzymatic, chemical or physical activators.

A prodrug (for example an enzyme) can be activated just prior to stinging capsule discharge by providing an activator compound (for example an ion), which can be diffused or pumped (during discharge) into the capsule. Alternatively, specific enzymes, molecules or pH conditions present in the target tissues, can activate the prodrug.

The cosmetic agent of the present invention can be, for example, an anti-wrinkling agent, an anti-acne agent, a vitamin, a skin peel agent, a hair follicle stimulating agent or a hair follicle suppressing agent. Using the stinging capsules of the present invention a more effective delivery of such cosmetic agents can be effected. Examples of cosmetic agents include, but are not limited to, retinoic acid and its derivatives, salicylic acid and derivatives thereof, sulfur-containing D and L amino acids and their derivatives and salts, particularly the N-acetyl derivatives, alpha-hydroxy acids, e.g., glycolic acid, and lactic acid, phytic acid, lipoic acid and many other agents which are known in the art, such as, for example the hair follicle stimulating or suppressing agents described hereinbelow.

In addition, dehydrated stinging capsules, which are capable of injecting a cosmetic dye, can be utilized as a needle in a pain free method of producing permanent or transient tattoos. For such purposes, a predetermined pattern of stinging capsules can be attached to a support such as a plaster, foil or the like as described hereinabove. The stinging capsules can be preloaded with a cosmetic dye or immersed therein prior to, or during triggering activation (e.g., the cosmetic dye can be applied to the skin). Upon stinging capsules discharge (via, for example, skin contact), the dye would penetrate into the skin to form a predetermined dye pattern (tattoo).

According to one preferred embodiment of the present invention, the therapeutic, diagnostic or cosmetic agent is disposed within the stinging capsule. In such a case, the capsule is loaded, prior to being desiccated, with the therapeutic, diagnostic or cosmetic agent, as described hereinabove.

As mentioned hereinabove, dehydrated stinging capsules can be triggered to discharge their tubules by their exposure to a rehydration substance. The exposure can be effected, for example, by dripping or spraying the solution onto the outer surface of the target tissue, or by placing a soaked membrane over the capsules. Upon activation, the immediate liquid surrounding the capsule is pumped into the capsule and than injected (into a tissue) via the tubule. Since the surrounding liquid is pumped into the capsules under extremely high pressures over a short period of time it is highly plausible that high molecular weight molecules, such as polypeptides polynucleotides and other complex molecules can penetrate the capsule and be delivered via the tubule upon discharge.

In any case, the dehydrated stinging capsules described above can be directly utilized to deliver the therapeutic, diagnostic or cosmetic agent into mammalian and other tissue by applying the dehydrated stinging capsules which include the agent, or by co-applying the agent and stinging capsules, onto a skin region of an individual (e.g. a human or livestock and other) followed by applying a rehydration substance to the skin region. Optional different orders by which the dehydrated stinging capsules, agents and rehydration substances of the present invention can be applied are summarized in Table 1 below.

TABLE 1
Optional applications of capsules, agent and rehydration substance

| | | Capsules | | | Rehydration |
|---|---|---|---|---|---|
| | Capsules | mixed | | Rehydration | substance |
| Capsules | pre-loaded | with | Agent | substance | mixed with |
| only | with agent | agent | only | only | agent |
| Step I | | | Step II | Step III | |
| Step I | | | | | Step II |
| | Step I | | | Step II | |
| | Step I | | | | Step II |
| | | Step I | | Step II | |
| | | Step I | | | Step II |
| Step II | | | Step I | Step III | |
| Step II | | | | | Step I |
| | Step II | | | | Step I |

Triggering activation of the dehydrated stinging capsules thus leads to the subsequent topical, transdermal/intradermal, transmucosal, transmembranal or transcuticular delivery of the therapeutic, diagnostic or cosmetic agent.

For topical, transmucosal or transnasal administration, the rehydration substance can be conveniently delivered in the form of drops or a spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount.

While capsule activation via exposure to a rehydration substance is preferred, the present invention also envisions including one or more triggering agents capable increasing efficiency and consistency of the capsules discharging activity.

Suitable triggering agents may be substances such as free and conjugated N-acetylated sugars or low molecular weight amino compounds which are known to be detected by at least two classes of stinging cell chemoreceptors. As described in U.S. Pat. No. 6,613,344, Sodium thiocyanate (NaSCN) is capable of triggering discharge of cnidocysts. In addition, Lubbock and Amos (1981) have shown that isolated cnida (cnidocysts) can discharge normally when placed in buffered EGTA or 10 mM citrate solution; Weber (1989) demonstrated the effect of dithioerthritol or proteases on discharging isolated cnida and Hidaka (1993) discussed various agents which can trigger cnida discharge.

Optionally, triggering of stinging capsules can be accelerated via providing to the capsules an electrical pulse, preferably of approximately 20-30 Volts for 30 microseconds (as is further described by Holstein and Tardent (1984) and Tardent and Holstein 1982).

For convenient storage and handling the dehydrated stinging capsules and the rehydration substance of the present invention may be packaged in a ready-to-use kit.

Thus, according to another aspect of the present invention there is provided a kit which includes a first container including a dehydrated stinging capsules component and a second container including a rehydration substance component. Preferably, the dehydrated stinging capsules component further includes a dry therapeutic, diagnostic or cosmetic agent being disposed within the first container. More preferably, a therapeutic, diagnostic or cosmetic agent is dissolved, or suspended, within the rehydration substance. Alternatively, the therapeutic, diagnostic or cosmetic agent of the kit may be included within a third container. The kit may also include instructions for use leaflet and a packaging material which identifies the kit for use in delivering the therapeutic, diagnostic or cosmetic agent to a tissue.

As mentioned hereinabove, the present invention can be utilized to deliver a variety of therapeutic agents. Such therapeutic agents combined with the effective delivery obtainable via capsules can be utilized to treat a variety of disorders.

An example of a very common skin infection is acne, which involve infestation of the sebaceous gland with P. acnes, as well Staphylococus aurus and pseudomonas. The disorder can be treated by anti-bacterial agents such as phenols, including cresols and resorcinols and antibiotics such as chloramphenicol, tetracyclines, synthetic and semi-synthetic penicillins, beta-lactames, quinolones, fluoroquinolnes, macrolide antibiotics, peptide antibiotics, cyclosporines, erytromycin and clindamycin.

Psoriasis, which is a common skin disorder can be treated by using the present invention for accurate and efficient intraepidermal delivery of steroidal anti-inflammatory agents or other known drugs with limited skin permeability.

Fungal infections can also be treated via the pharmaceutical composition of the present invention. Superficial fungal infection of the skin is one of the commonest skin diseases seen in general practice. Dermatophytosis is probably the most common superficial fungal infection of the skin. Candidiasis is an infection caused by the yeast like fungus candida albicans or occasionally other species of candida. Antifungal drugs, which are active against dermatophytes and candida such as azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration can be delivered intraepidermally via the method of the present invention.

The present invention can be also used for delivering pigments, such as photosensitizers utilizable in photo dynamic therapy (PDT), into cells of skin cancer or other skin disorders. Photosensitizers are chemical compound which produce a biological effect upon photoactivation, or a biological precursor of a compound that produces a biological effect upon photoactivation. Examples of photosensitizers which can be delivered by the stinging capsules of the present invention include, but are not limited to, hematoporphyrins (Batlle 1993 J. Photochem. Photobiol. Biol. 20:5-22 and Kessel 1988 Cancer Let. 39:193-198), uroporphyrins and phthalocyanines (Kreimer-Birnbaum, 1989 Seminars in Hematology 26:157-173), purpurins (Morgan et al. 1990 Photochem. Photobiol. 51:589-592 and Kessel, 1989 Photochem. Photobiol. 50:169-174), acridine dyes and bacteriochlorophylls (Beems et al. 1987 Photochem. Photobiol. 46:639-643 and Kessel et al. 1989 Photochem. Photobiol. 49:157-160), and bacteriochlorins (Gurinovich et al. 1992 J. Photochem. Photobiol. Biol. 13:51-57).

By enabling accurate and efficient delivery of photosensitizers, the present invention substantially improves the efficiency of PDT.

Eye infections such as conjuctivitis, caused by bacteria such as staphylococcus aureus, streptococcus pneumoniae, and haemophilus influenzae can be treated with antibiotic ointments, e.g., bacitracin which is delivered via the method of the present invention.

Chronic rheumatic or arthritic conditions are usually treated by NSAIDs. Such as salicylic acid, or aspirin, and ibuprofen are well-known examples of NSAI drugs. Patients taking NSAIDs drugs orally face an increased risk for peptic ulcers and gastrointestinal blood loss resulting in anaemia. Such adverse reactions especially plague patients taking NSAIDs drugs over prolonged periods. Transdermal administration of NSAIDs via the delivery device or method of the present invention will prevent the gastrointestinal complications. Transdermal drug delivery according to the present invention provides other benefits such as less frequent dosing; better controlled drug release, and a greater ability to target delivery to specific tissue sites.

Anaesthetics can be used for alleviating pain for example during suturing, or in infections, which are accompanied with pain sensation. Examples of topical anaesthetic drugs include without limitation benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof all of which are deliverable via the delivery device or method of the present invention.

The present invention can be used to treat hair loss, excessive hair growth, or discoloration of the hair.

For example, a hair follicle stimulating agent such as hinokitiol, or pantothenic acid can be delivered by the stinging capsules of the present invention directly into the follicle in order to stimulate hair growth.

Allternatively, the dehydrated stinging capsule composition of the present invention can be utilized to deliver, directly into hair follicles, a follicle suppressing agent capable of suppressing hair growth. Examples of agents capable of suppressing hair growth include, but are not limited to, non-steroidal suppressors of angiogenesis and inhibitors of 5-alpha reductase, ornithine decarboxylase, S-adenosylmethionine decarboxylase, gamma-glutamyl transpeptidase, and transglutaminase.

The present invention can also be utilized to pigment hair color by delivering, for example, melanin or tyrosinase, into the hair follicle.

In such cases, the present invention can be utilized to deliver drugs such as hormones (e.g., insulin), antibiotics, cardiac drugs and the like.

The dehydrated stinging capsules of the present invention can also be utilized for vaccination. Vaccine antigens can be delivered to specialized immune cells underlying the skin or into blood circulation (as described above).

Absorption into the blood stream following transdermal delivery will most likely result in transport of the antigen to the phagocytic cells of the liver, spleen, and bone marrow. Since such cells serve as antigen presenting cells, a strong immunogenic response will be elicited leading to effective immunization.

Thus, the present invention overcomes the limitations of prior art devices and methods by providing novel compositions and kits including dehydrated stinging capsules which are highly stable in storage and which can be readily utilized for rapid, safe, efficient and convenient delivery of agents across epidermal mucousal membranal and/or cuticle barriers.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

### Example 1

### Freeze Dried Stinging Capsules

Materials and Methods:

Isolation of capsules: Fresh tissue of the sea anemone Aiptasia diaphana was homogenized in sodium citrate as described by Salleo et al. (Physiol Zool 61: 272-279, 1988). The homogenate (300 μl) was added to percol (300 μl) in a microfuge tube. The tube was shaken over ice for 30 min and then centrifuged for 10 minutes, at 1000 rpm. The pellet was washed 3 times with H₂O and re-suspended in 50 μl H₂O as a liquid preparation.

Freeze drying: Isolated capsules were suspended in 30 mM NaCl solution (10⁶ capsules/ml) and dispensed in Eppendorf tubes. The tubes were dipped in liquid nitrogen then subjected to freeze drying over night. The freeze dried capsules were kept as a dry powder until used.

Testing of the stinging (discharging) capacity of capsules: samples of freeze-dried and non-treated (control) capsule preparations were applied to a microscope slide followed by adding sodium thiocyanate (NaSCN, 2 μl). An immediate tubules discharge from capsules, observed under a light microscope (Leitz Laborlux S), was indicative of a positive activity of capsules.

Results:

Activation and stability of isolated capsules: fresh capsules were triggered by exposure to 1% SDS solution resulting in about 80% activation within 1 minute.

All isolated capsules stored in water discharged their tubules within 20 hours at room temperature.

Activation and stability of freeze-dried capsules: freeze-dried capsules were triggered by rehydration in a saline or 1% SDS solution resulting in over 90% activation within 20 seconds.

Freeze dried stinging capsule preparations which were stored for one year at 4° C., or for six month at room temperature, or for one month at 40° C., remained intact and capable of discharging tubules immediately upon rehydration.

### Example 2

### Desiccated Stinging Capsules

Materials and Methods:

Isolation of capsules: as described in Example 1 above.

Desiccation: isolated capsules were suspended in an organic solvent (ethanol, propylene glycol, or octyl palminate) then centrifuged at 1000 g for 10 minutes. The resulting pellets were kept at 4° C. re-suspended in the organic solvent and centrifuged repeatedly for 3 times. The organic solvent was replaced every day for 3 days and the resulting desiccated capsules were either kept in the same organic solvent or formulated in ethanol 2% hydroxy propyl cellulose until used.

Testing of the stinging (discharging) capacity of desiccated capsules: samples of desiccated and non-treated (control) capsule preparations were applied to a microscope slide followed by adding sodium thiocyanate (NaSCN, 2 μl). An immediate tubules discharge from capsules, observed under a light microscope (Leitz Laborlux S), was indicative of a positive activity of capsules.

Results:

All non desiccated capsules which were kept at 4° C. in water discharged their tubules within 20 hours. On the other hand, all desiccated capsules remained intact and did not discharge their tubules during one month of storage at 4° C. Following storage, the desiccated capsules were triggered by rehydration in a saline or 1% SDS solution resulting in over 90% activation within 20 seconds.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications disclosed therein and/or mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1. Anderson, C. L., Canning, E. U., and Okamura, B. (1998). “A triploblast origin for Myxozoa?” Nature, 392(6674), 346-7.

2. Brennecke, T., Gellner, K., and Bosch, T. C. (1998). “The lack of a stress response in Hydra oligactis is due to reduced hsp70 mRNA stability.” Eur J Biochem, 255(3), 703-9.

3. Godkenecht, A., and Tardent, P. (1988). “Discharge and mode of action of the tentacular nematocysts of Anemonia sulcata (Antozoa: Cnidaria).” Marine Biology, 100, 83-92.

4. Heeger, T., Moller, H., and Mroweitz, U. (1992). “Protection of human skin against jellyfish (Cyanea capillata) stings.” Marine Biology, 113, 669-678.

5. Hidaka, M. (1992). “Effects of Ca+ on the volume of nematocysts isolated from acontia of the sea anemone Calliactis tricolor.” Comp Biochem Physiol, 101A(4), 737-741.

6. Hidaka, M. (1993). “Mechanism of nematocystn discharge and its cellular control.” Advances in Comparative and Environmemtal Physiology, 15, 45-76.

7. Holstein, T., and Tardent, P. (1984). “An ultrahigh-speed analysis of exocytosis: nematocyst discharge.” Science, 223(4638), 830-3.

8. Lotan, A., Fishman, L., Loya, Y., and Zlotkin, E. (1995). “Delivery of a nematocyst toxin Nature, 375(6531), 456.

9. Lotan, A., Fishman, L., and Zlotkin, E. (1996). “Toxin compartmentation and delivery in the Cnidaria: the nematocyst's tubule as a multiheaded poisonous arrow.” J Exp Zool, 275(6), 444-51.

10. Lubbock, R. (1979). “Chemical recognition and nematocyte exitation in sea anemone.” J. exp. Biol., 83, 283-292.

11. Lubbock, R., and Amos, W. B. (1981). “Removal of bound calcium from nematocyst contents causes discharge.” Nature, 290(5806), 500-1.

12. Mariscal, R. N. (1974). Coelenterate biology: reviews and new perspectives, Academic Press, New York.

13. Siddall, M. E., Martin, D. S., Bridge, D., Desser, S. S., and Cone, D. K. (1995). “The demise of a phylum of protists: phylogeny of Myxozoa and other parasitic cnidaria.” J Parasitol, 81(6), 961-7.

14. Smothers, J. F., von Dohlen, C. D., Smith, L. H., Jr., and Spall, R. D. (1994). “Molecular evidence that the myxozoan protists are metazoans.” Science, 265(5179), 1719-21.

15. Tardent, P. (1995). “The cnidarian cnidocyte, a high-tech cellular weaponry.” BioEssays, 17(4), 351-362.

16. Tardent, P., and Holstein, T. (1982). “Morphology and morphodynamics of the stenotele nematocyst of Hydra attenuata Pall. (Hydrozoa, Cnidaria).” Cell Tissue Res, 224(2), 269-90.

17. Thorington, G. U., and Hessinger, D. A. (1988). “Control of cnida discharge: I. evidence for two classes of chemoreceptor.” Biol. Bull., 174, 163-171.

18. Watson, G. M., and Hessinger, D. (1989). “Cnidocyte mechanoreceptors are tuned to the movements of swimming prey by chemoreceptors.” Science, 243, 1585-1591.

19. Watson, G. M., and Hessinger, D. A. (1992). “Receptors for N-acetylated sugars may stimulate adenylate cyclase to sensitize and tune mechanoreceptors involved in triggering nematocyst discharge.” Exp Cell Res, 198(1), 8-16.

20. Weber, J. (1989). “Nematocysts (stinging capsules of Cnidaria) as Donnan-potential-dominated osmotic systems.” Eur J Biochem, 184(2), 465-76.

21. Westfall, J. A., Bradbury, P. C., and Townsend, J. W. (1983). “Ultrastructure of the dinoflagellate Polykrikos. I. Development of the nematocyst-taeniocyst complex and morphology of the site for extrusion.” J Cell Sci, 63, 245-61.

What is claimed is:

1. A dry composition-of-matter comprising at least one dehydrated stinging capsule.

2. The dry composition-of-matter of claim 1, wherein a moisture content of the composition-of-matter is less than 10% by weight.

3. The dry composition-of-matter of claim 1, further comprising a therapeutic, diagnostic or cosmetic agent.

4. The dry composition-of-matter of claim 3, wherein said therapeutic, diagnostic or cosmetic agent is disposed within said at least one dehydrated stinging capsule.

5. The dry composition-of-matter of claim 3, wherein said cosmetic agent is selected from the group consisting of a cosmetic dye, an anti wrinkling agent, an anti-acne agent, a vitamin, a skin peel agent, a hair follicle stimulating agent and a hair follicle suppressing agent.

6. The dry composition-of-matter of claim 3, wherein said therapeutic agent is selected from the group consisting of a drug, a nucleic acid construct, a vaccine, a hormone, an enzyme and an antibody.

7. The dry composition-of-matter of claim 3, wherein said therapeutic agent is a prodrug activatable prior to, during or following discharge of said at least one dehydrated stinging capsule.

8. The dry composition-of-matter of claim 3, wherein said at least one dehydrated stinging capsule is capable of delivering said therapeutic, diagnostic or cosmetic agent into a tissue.

9. The dry composition-of-matter of claim 1, wherein an endogenous toxin naturally stored within said at least one dehydrated stinging capsule is substantially non-toxic to mammals.

10. The dry composition-of-matter of claim 9, wherein said endogenous toxin is non-functional.

11. The dry composition-of-matter of claim 1, wherein said at least one dehydrated stinging capsule is derived from an organism of a class selected from the group consisting of Anthozoa, Hydrozoa and Scyphozoa.

12. The dry composition-of-matter of claim 1, wherein said at least one dehydrated stinging capsule is derived from an organism of a phylum selected from the group consisting of Cnidaria, Dinoflagellata and Myxozoa.

13. A kit, comprising a first component including at least one dehydrated stinging capsule and a second component including a rehydration substance capable of activating said at least one dehydrated stinging capsule.

14. The kit of claim 13, wherein said rehydration substance is a buffer solution.

15. The kit of claim 13, wherein said rehydration substance includes at least one triggering agent.

16. The kit of claim 13, wherein said rehydration substance includes a therapeutic, diagnostic or cosmetic agent.

17. The kit of claim 13, wherein an endogenous toxin stored within said at least one dehydrated stinging capsule is substantially non-toxic to mammals.

18. The kit of claim 17, wherein said endogenous toxin is non-functional.

19. The kit of claim 13, wherein said at least one dehydrated stinging capsule is derived from an organism of a class selected from the group consisting of Anthozoa, Hydrozoa and Scyphozoa.

20. The kit of claim 13, wherein said at least one dehydrated stinging capsule is derived from an organism of a phylum selected from the group consisting of Cnidaria, Dinoflagellata and Myxozoa.

21. The kit of claim 13, further comprising a third component including a therapeutic, diagnostic or cosmetic agent.

22. The kit of claim 21, wherein said cosmetic agent is selected from the group consisting of a cosmetic dye, an anti-wrinkling agent, an anti-acne agent, a vitamin, a skin peel agent, a hair follicle stimulating agent and a hair follicle suppressing agent.

23. The kit of claim 21, wherein said therapeutic agent is selected from the group consisting of a drug, a nucleic acid construct, a vaccine, a hormone, an enzyme and an antibody.

24. The kit of claim 21, wherein said therapeutic agent is a prodrug activatable prior to, during or following discharge of said at least one dehydrated stinging capsule.

25. The kit of claim 21, wherein said at least one dehydrated stinging capsule is capable of delivering said therapeutic, diagnostic or cosmetic agent into a tissue.

26. The kit of claim 13, wherein said first component further comprising a therapeutic, diagnostic or cosmetic agent being disposed within said at least one dehydrated stinging capsule.

27. The kit of claim 13, further comprising an instructions-for-use leaflet and a packaging material which identifies the kit for use in delivering a therapeutic, diagnostic or cosmetic agent to a tissue.

28. A method of producing dehydrated stinging capsules, comprising subjecting a stinging capsule preparation to desiccating conditions, to thereby producing the dehydrated stinging capsules.

29. The method of claim 28, wherein said desiccating conditions include freezing said stinging capsule preparation.

30. The method of claim 29, wherein said freezing is effected under vacuum conditions.

31. The method of claim 30, wherein said desiccating conditions include exposure to a substance capable of absorbing moisture from said stinging capsules.

32. The method of claim 31, wherein said substance is selected from the group consisting of ethanol, propylene glycol, acetyl palmitate oil, and a salt.

33. The method of claim 28, wherein said stinging capsules are derived from an organism of a class selected from the group consisting of Anthozoa, Hydrozoa and Scyphozoa.

34. The method of claim 28, wherein said stinging capsules are derived from an organism of a phylum selected from the group consisting of Cnidaria, Dinoflagellata and Myxozoa.

35. A method of delivering a therapeutic, diagnostic or cosmetic agent into a tissue, comprising:
(a) applying to the tissue at least one dehydrated stinging capsule and the therapeutic, diagnostic or cosmetic agent; and
(b) activating a discharge of said at least one dehydrated stinging capsule to thereby deliver the therapeutic, diagnostic or cosmetic agent into the tissue.

36. The method of claim 35, wherein an endogenous toxin stored within said at least one dehydrated stinging capsule is substantially non-toxic to mammals.

37. The method of claim 36, wherein said endogenous toxin is non-functional.

38. The method of claim 35, wherein said at least one dehydrated stinging capsule is derived from an organism of a class selected from the group consisting of Anthozoa, Hydrozoa and Scyphozoa.

39. The method of claim 35, wherein said at least one dehydrated stinging capsule is derived from an organism of a phylum selected from the group consisting of Cnidaria, Dinoflagellata and Myxozoa.

40. The method of claim 35, wherein said therapeutic agent is selected from the group consisting of a drug, a nucleic acid construct, a vaccine, a hormone, an enzyme and an antibody.

41. The method of claim 35, wherein said therapeutic agent is a prodrug activatable prior to, during or following discharge of said at least one dehydrated stinging capsule.

42. The method of claim 35, wherein said cosmetic agent is selected from the group consisting of a cosmetic dye, an anti-wrinkling agent, a vitamin, a skin peel agent, a hair follicle stimulating agent and a hair follicle suppressing agent.

43. The method of claim 35, wherein step (b) is effected by exposing said at least one dehydrated stinging capsule to a rehydration substance.

44. The method of claim 43, wherein said rehydration substance is a buffer solution.

45. The method of claim 43, wherein said rehydration substance includes at least one triggering agent.

46. The method of claim 43, wherein said rehydration substance includes a therapeutic, diagnostic or cosmetic agent.
